(19) United States
(12) Patent Application Publication
(10) Pub. No.: US 2015/0069164 A1
(43) Pub. Date: Mar. 12, 2015

(54) Retractable Medical Tubing Reel

(71) Applicant: Lifeline-Reel LLC, Spokane, WA (US)

(72) Inventor: David James Moore, Spokane, WA (US)

(73) Assignee: Lifeline-Reel LLC, Spokane, WA (US)

(21) Appl. No.: 14/023,312

(22) Filed: Sep. 10, 2013

(51) Int. Cl. A61M 39/08; B65H 75/44

(52) U.S. Cl. CPC A61M 39/08; B65H 75/4428; A61M 2039/087; A61M 2202/0208; A61M 2202/0283; A61M 2209/08; B65H 2701/33
USPC 242/382; 137/355.28

(57) ABSTRACT

This disclosure describes, in part, a reel assembly configured to store and automatically retract flexible medical tubing. The reel assembly may include a constant torque spring configured to bias a rotational force on a spool. The spool may have clusters of teeth alternative with tooth-less areas. The reel assembly may also comprise a pawl with an internal spring configured to interact with the clusters of teeth and tooth-less areas in order to prevent/permit retracting a portion of fed medical tubing by the constant torque spring.

### BACKGROUND

Generally, people who require oxygen therapy for a medical condition must constantly carry an oxygen supply. In many case, when such a person is within a home or hospital room they are limited to a small area or radius around the oxygen supply due to a limited amount of medical tubing supplying the oxygen. Additionally, every year people who need oxygen therapy are injured in trip and fall accidents involving the medical tubing supplying the oxygen. These accidents may involve the medical tubing becoming tangled in the person's feet. Moreover, the likelihood of a trip and fall accident increases as the person needs a greater length of medical tubing to move around a home and/or hospital room. Accordingly, there remains a need for an improved reel assembly and methods which allow for easily retractable access to a greater length of medical tubing and which may prevent needless accidents caused by excess medical tubing.

### BRIEF DESCRIPTION OF THE DRAWINGS

The detailed description is described with reference to the accompanying figures. In the figures, the left-most digit(s) of a reference number identifies the figure in which the reference number first appears. The use of the same reference numbers in different figures indicates similar or identical components or features.

FIG. 1 illustrates a perspective view of a first surface of an example automatically retractable medical tubing reel.

FIG. 2 illustrates a first surface view of the example automatically retractable medical tubing reel shown in FIG. 1.

FIG. 3 illustrates a view of the bottom surface of the example automatically retractable medical tubing reel shown in FIG. 1.

FIG. 4 illustrates a first surface view of the top of the example automatically retractable medical tubing reel shown in FIG. 1 with an exposed intake connector.

FIG. 5 illustrates a second surface view of the example automatically retractable medical tubing reel shown in FIG. 1.

FIG. 6 illustrates a second surface view of the example automatically retractable medical tubing reel shown in FIG. 1 with an exposed constant torque spring mechanism.

FIG. 7 illustrates a side surface view of the example automatically retractable medical tubing reel shown in FIG. 1. including medical tubing intake/outtake opening 210.

FIG. 8 illustrates a perspective view of the example automatically retractable medical tubing reel shown in FIG. 1 with a first half removed to expose the spool mechanism.

FIG. 9 illustrates a second surface view of the example automatically retractable medical tubing reel shown in FIG. 1 with a second half removed and a transparent spool mechanism.

FIG. 10 illustrates a perspective view of the constant torque spring mechanism as coupled to spool mechanism.

FIG. 11 illustrates a first surface view of an example automatically retractable medical tubing reel configured without clips.

FIG. 12 illustrates an alternative example automatically retractable medical tubing reel.

FIG. 13 illustrates an example flow diagram of a process for automatically retracting a portion of medical tubing using an automatically retractable medical tubing reel.

### DETAILED DESCRIPTION

### Overview

This disclosure describes, in part, a reel assembly configured to store a portion of medical tubing and feed the medical tubing as desired or requested by a user. The reel assembly may also be configured with a spring mechanism and ratchet system to automatically retract the fed portion of medical tubing. For example, the reel assembly may store the medical tubing while the user is stationary. The reel assembly may allow a substance (e.g., oxygen gas, nitrous oxide, albuterol sulfate, other compressed gases or medications) to pass from a source (e.g., portable tank, stationary cylinder, wall unit, ceiling unit, nebulizer unit, etc.) through the stored medical tubing to the user. Additionally, in some implementations, when the user moves to a different location relative to the reel assembly and pulls on the medical tubing, tubing is delivered from the assembly allowing the user to still receive an uninterrupted supply of the substance without requiring the user to transport the source. In some implementations, once the user moves to a position nearer the substance source, the user may manipulate the tubing such that the reel assembly may mechanically recall the previously delivered tubing.

In some implementations, the reel assembly (hereinafter, “reel”) may be removably mounted to a surface (e.g., a wall, a ceiling, a floor or the like). In some implementations, the mount may allow the reel to swivel at least 180 degrees. In other implementations, the mount may allow the reel to swivel 360 degrees. For example, as the user moves from one location to another location the reel may rotate relative to the mount to track or follow the user's movement through a room.

In some implementations, the reel may have an internal spool configured to store medical tubing of various compositions (e.g., latex-free polyvinyl chloride (PVC), vinyl, latex). In some implementations, the spool may be configured to store medical tubing of various gauges (e.g., from about 0.005 inches to about 0.350 inches). In some implementations, the spool may be configured to store medical tubing of a length of about 20 feet to about 200 feet. In some implementations, the medical tubing stored in the reel may have a kink-resistant and/or crush-resistant lumen to resist occlusion.

The reel may have a spring mechanism attached to the spool biased to maintain a position of the spool in a stored position. In some implementations, the spool may have a plurality of teeth or projections to interact with a ratchet mechanism or pawl. The ratchet mechanism may be configured to catch one of the plurality of teeth as the users pulls the medical tubing way from the reel. In some implementations, the ratchet mechanism may be configured to disengage from a caught tooth when the user lightly pulls on the medical tubing. In this implementation, the spring mechanism may be configured to rotate the spool such that the medical tubing is retracted by the spool of the reel.

The assembly for automatically retracting medical tubing may be implemented in many ways. Example implementations are provided below with reference to the figures.

### Example Retractable Medical Tubing Reel

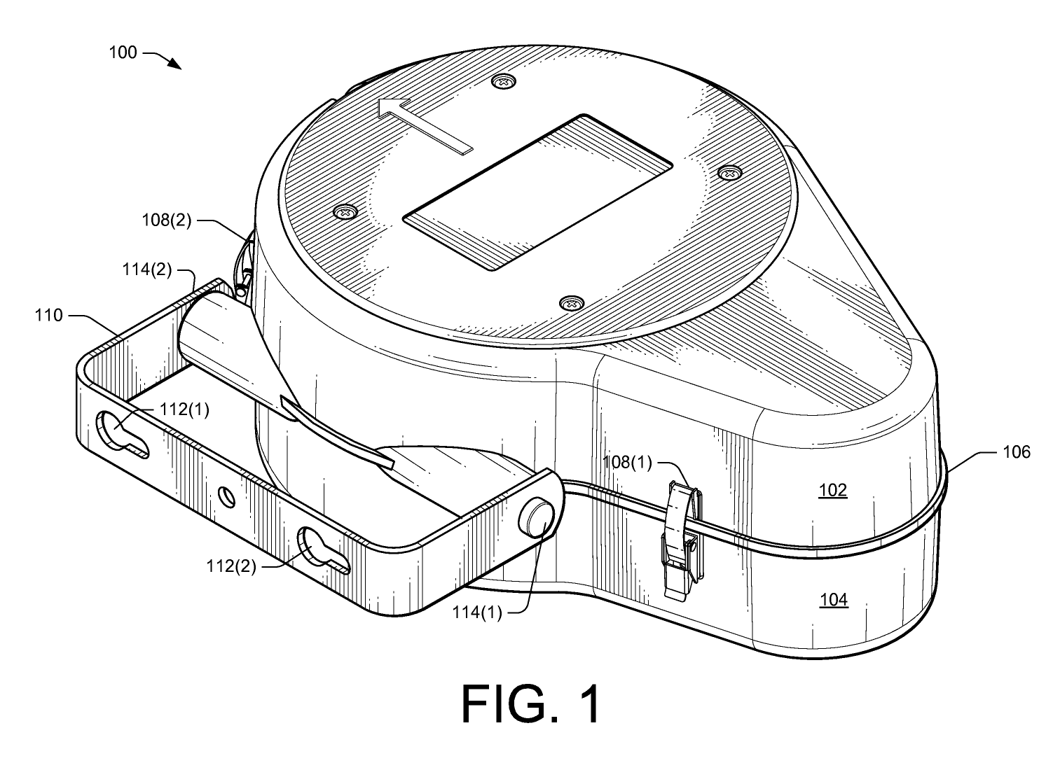

FIG. 1 illustrates a perspective view of a first surface of an automatically retractable medical tubing reel 100. In some implementations, the reel 100 may include a first half 102 coupled to a second half 104 forming a case. In some implementations, second half 104 may include a ridge 106 configured to receive and secure an edge of the first half 102. The first half 102 and second half 104 of reel 100 may be composed of, for example, polyethylene terephthalate, high density polyethylene, polyvinyl chloride, low density polyethylene, polypropylene, polystyrene, and polycarbonate or any other suitable material. Additionally or alternatively, the half 102 and second half 104 of reel 100 may be composed of metal, wood, glass, stone, ceramic or the like.

In some implementations, the first half 102 and second half 104 of reel 100 may be fastened by a plurality of clips 108(1)-(N). However, as described below, other attachment mechanisms may be implemented.

FIG. 1 also illustrates that reel 100 may include a mounting bracket 110 with a plurality of mounting hardware holes 112(1) and 112(2). In some implementations, mounting bracket 110 may be mounted to a ceiling, a wall, a floor, or any other stable structure. Mounting bracket 110 may be composed of various metals such as, for example, aluminum, aluminum alloys, steel, or the like. In some implementations, mounting bracket 110 may be painted or otherwise coated to resist corrosion. In some implementations, each end of mounting bracket 110 may be joined or connected to the reel 100 via pegs 114(1) and 114(2) (not shown) projecting parallel to the first half 102 of the case. Pegs 114(1) and peg 114(2) may allow the reel 100 to rotate relative to mounting bracket 110 once fastened. In some implementations, reel 100 may be configured to rotate at least 180°.

In other implementations, mounting bracket may be configured to attach to a single attachment point on reel 100. In some implementations, the single attachment point may be a spherical projection upon which the mounting bracket may be attached (e.g., pressure fit). In this implementation, the reel 100 may be configured to rotate in at least 360° relative to a mounting surface.

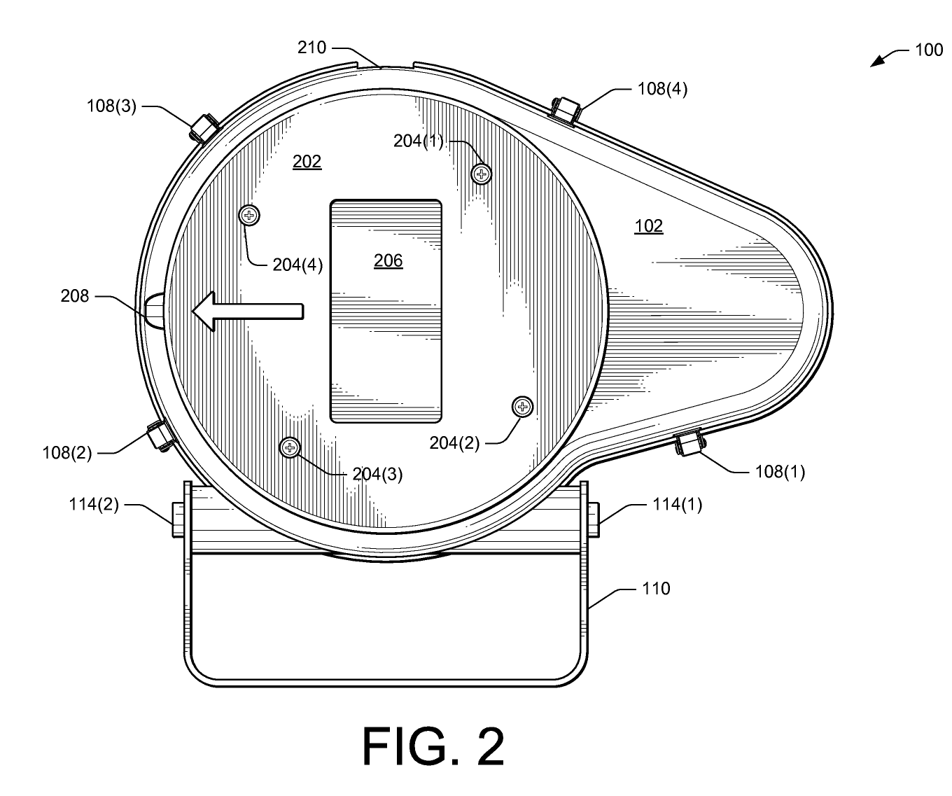

FIG. 2 illustrates a first surface view of first half 102 of reel 100. Furthermore, FIG. 2 illustrate four clips 108(1)-(4) configured to hold the first half 102 of reel 100 to the second half 104 of reel 100. In some implementations, first half 102 may have a cover 202 configured to cover a medical tubing intake connector (discussed below). In some implementations, cover 202 may be composed of the same material as the first half 102 of reel 100. In other implementations, cover 202 may be composed of a material different from the first half 102 of reel 100.

Cover 202 may be secured by a plurality of fasteners 204(1)-(4). In some implementations, cover 202 may be removable from reel 100 while first half 102 remains in place. In some implementations, cover 202 may include a label area 206 to identify a substance transported through the attached medical tubing (i.e., oxygen (O₂) gas), manufacture information, or the like.

In some implementations, first half 102 may include intake port 208. Intake port 208 may provide a secure pathway allowing medical tubing of various gauges to be connected to reel 100. As illustrated, intake port 208 may be an open channel on the top surface of the first half 102. In another implementations, intake port 208 may be formed as a hole through the first half 102 of reel 100. In other implementations, intake port 208 may be formed as a hole through cover 202. In yet another implementation, intake port 208 may be formed as a hole through the second half 104 of reel 100.

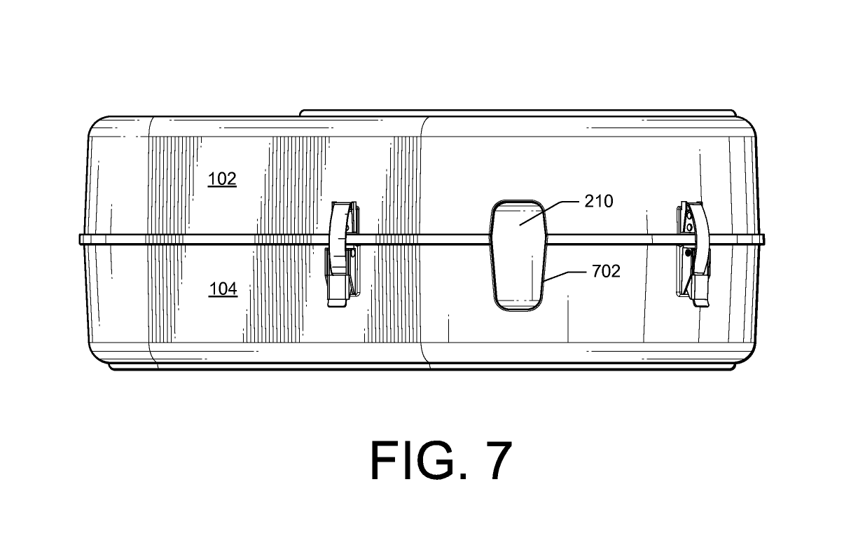

FIG. 2 further illustrates that reel 100 may also include medical tubing intake/outtake opening 210. In some implementations, intake/outtake opening 210 may provide an opening for the medical tubing to exit the reel 100 and, ultimately, interact with a user. In other implementations, the intake/outtake opening 210 may provide an opening for the medical tubing retract onto a spool housed within reel 100. Intake/outtake opening 210 will be described in greater detail below with regard to FIG. 7.

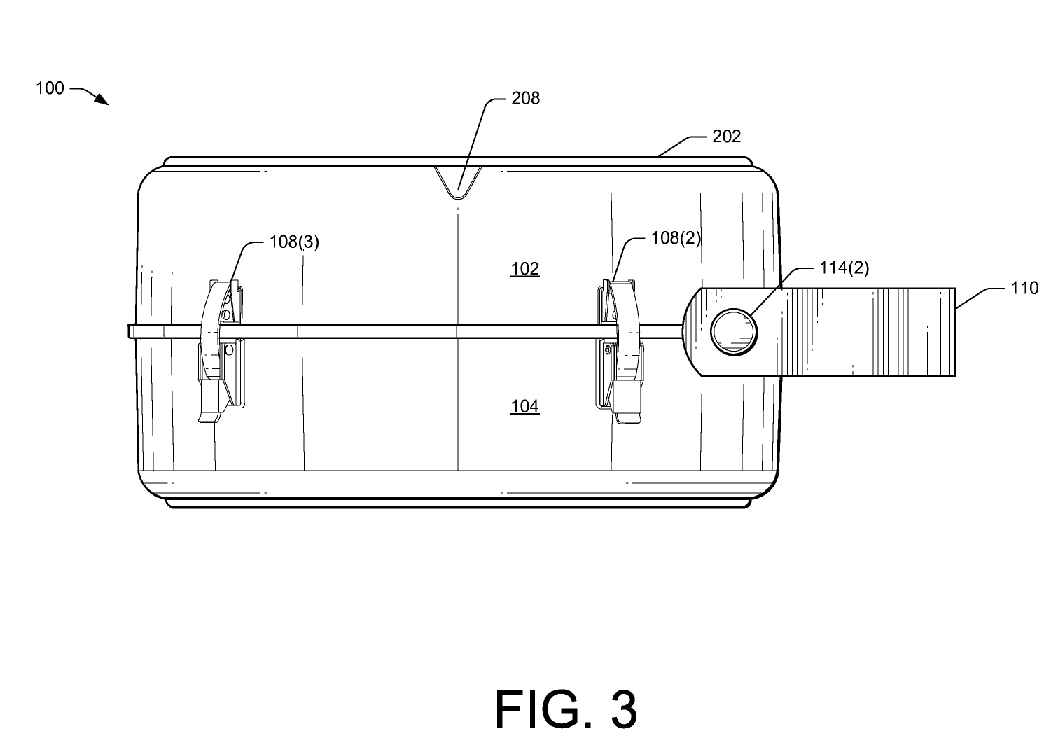

FIG. 3 illustrates a view of the bottom surface of reel 100 showing intake port 208. As illustrated intake port 208 may be formed as a channel within the first half 102 and closed on the top by cover 202. In some implementations, intake port 208 may be larger or smaller to accommodate medical tube of various diameters or gauges.

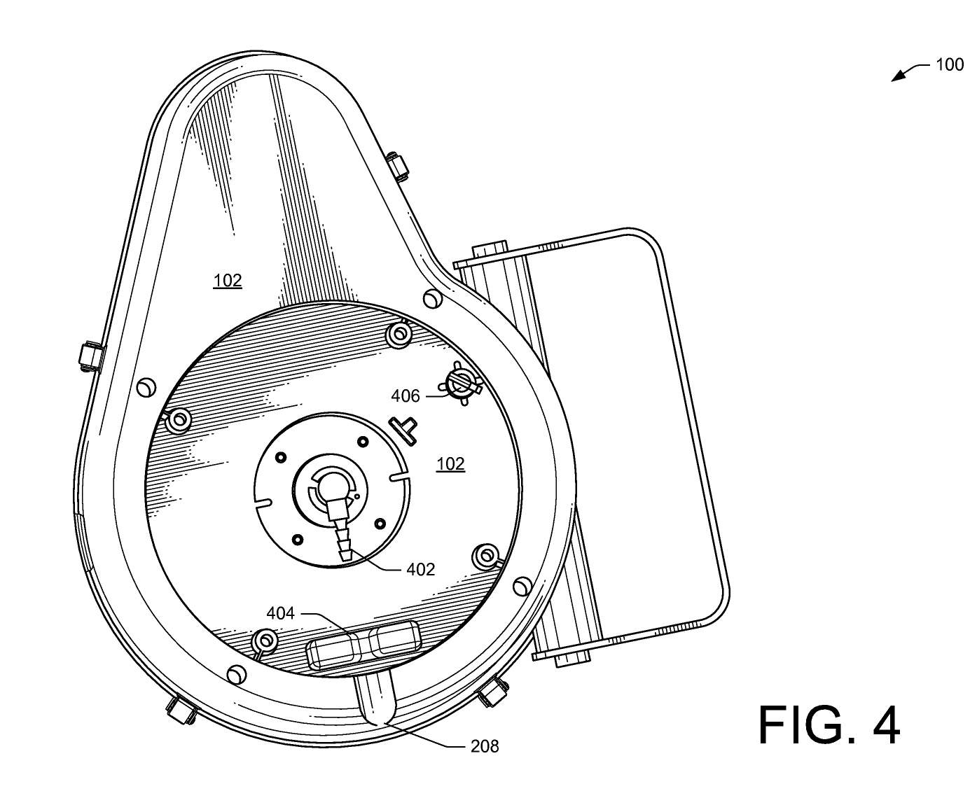

FIG. 4 illustrates a first surface view of reel 100 with cover 202 removed from the first half 102. In some implementations, removal of cover 202 may reveal a recessed portion of the first half 102. As mentioned above, removal of cover 202 may expose medical tubing intake connector 402. Intake connector 402 may be configured to secure an end of medical tubing. For example, intake connector 402 may provide an intake point for connection of medical tubing originating at the substance source (e.g., portable tank, stationary cylinder, wall oxygen unit, etc). In some implementations, intake connector 402 may be composed of medical grade material such, for example, a plastics, silicones, latex-free PVC, etc.

Intake connector 402 is shown as barbed or with a progression of overlapping conical shapes from smaller at the opening to larger at the base in order to secure medical tubing of various sizes. However, other connectors are envisioned, such as, for example, quick connect hose fitting, push fit fittings, push lock fittings, etc.

In some implementations, intake connector 402 may be stationary relative to the movement of the spool. In some implementations, reel 100 may include a medical tubing guide 404 which may guide medical tubing entering intake port 208 toward intake connector 402. In some implementations, tubing guide 404 may provide an additional clamp or securing mechanism configured to hold the tubing on intake connector 402.

FIG. 4 illustrates pawl holder 406 on the recessed portion of the first half 102. As will be discussed below, pawl holder 406 may be configured to secure a pawl in relation to the spool. In some implementations, pawl holder 406 may be molded as part of the first half 102. In other implementations, pawl holder 406 may include additional hardware (i.e., springs, coils elastic bands) in order to give the pawl a spring effect.

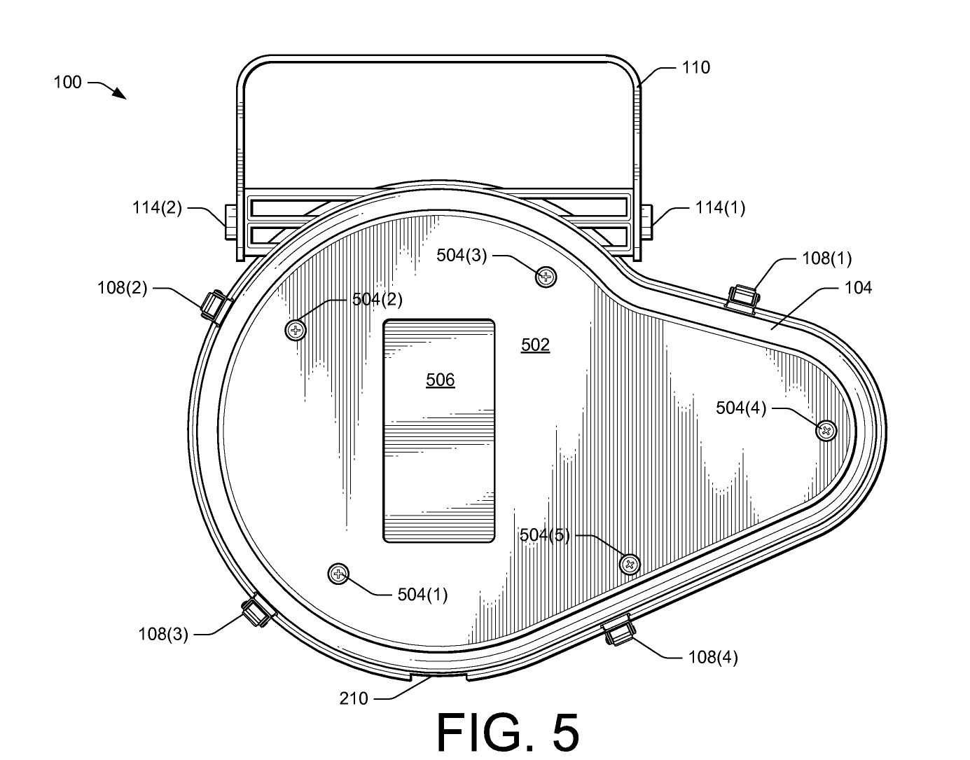

FIG. 5 illustrates a second surface view of reel 100 and, specifically, the second half 104. As illustrated, second half 104 may include a cover 502. In some implementations, the cover is removable by, for example, removing fasteners 504(1)-(5). Fasteners 504(1)-(5) are illustrated as Phillips head screws; however, other fasteners are envisioned. In other implementations, second half 104 may be configured without cover 502.

In some implementations, cover 502 may include a label area 506 to identify a substance transported through the attached medical tubing (i.e., oxygen (O₂) gas), manufacture information, or the like. In some implementations, label area 506 may include the same identification as included in label area 206. However, in other implementation, label area 506 may include information different from label area 206.

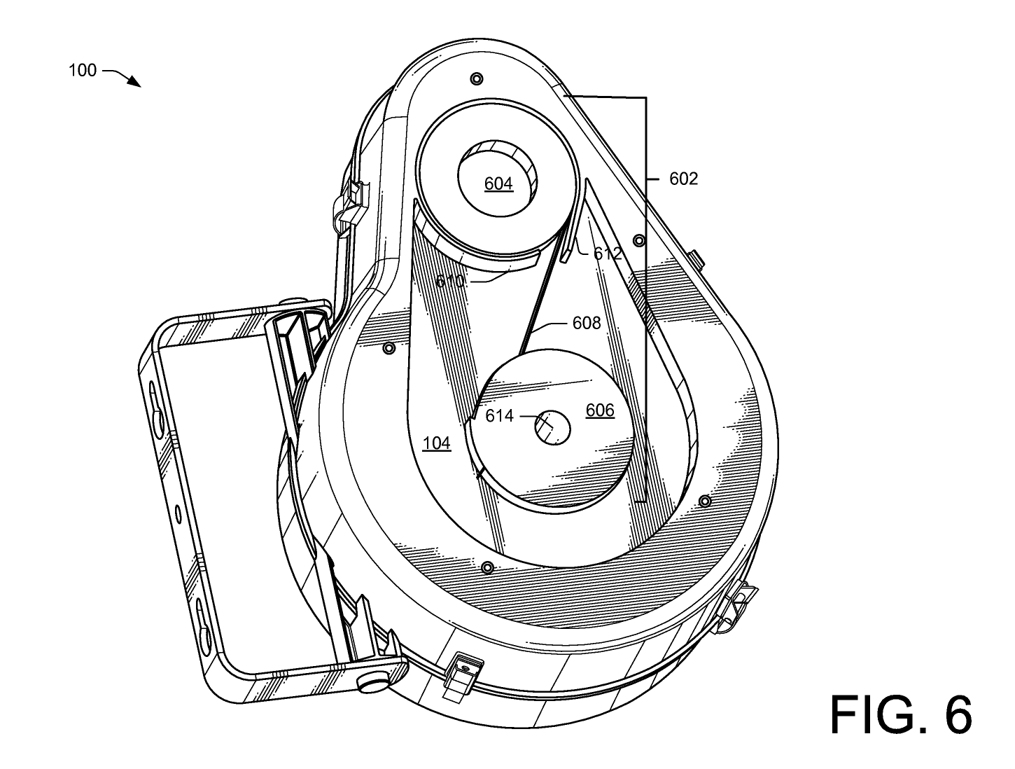

FIG. 6 illustrates a second surface view of reel 100 with cover 502 removed to expose a recessed area of second half 104. Furthermore, the recessed area of second half 104 includes a constant torque spring mechanism 602. The constant torque spring mechanism 602 may include output drum 604 and storage drum 606. In some implementations, the output drum 604 may include a ribbon of spring steel 608 wound into a coil. In some implementations, the ribbon of spring material may be made of, for example, high-carbon steel, nickel-chromium alloys, or the like. In some implementations, the ribbon of spring steel 608 may be confined by barrier 610 and ribbon guide 612. In some implementations, the barrier 610 and ribbon guide 612 may be molded as part of the second half 104 of reel 100.

In some implementations, the ribbon of spring steel 608 may be guided by the ribbon guide 612 to counter wind on the storage drum 606. For example, the spring steel 608 may be coiled around the storage drum 606 in a coiling direction opposite of the coiling direction on the output drum 604 as a spool of reel 100 is rotated to feed medical tubing to a user. An end of the ribbon of spring steel may be permanently secured to the storage drum by any number of mechanisms (e.g., a rivet, captive fastener, set screw, etc). In some implementations, the storage drum 606 may be a constructed of a lightweight durable material (e.g., aluminum, plastics, etc). In some implementations, as discussed below, the spring steel 608 is configured to remain on the output drum 604 while at rest. In some implementations, a constant force or resistance is creating while counter winding the spring steel 608 from the output drum 604 to the storage drum 606.

In some implementations, the torque or tension required to manipulate the ribbon of spring steel 608 from the output drum 604 to the storage drum may be variable. For example, in some implementations, it may be desirable to have minimal tension required so a user can easily pull the medical tubing from the spool. In some implementations, the torque and/or tension required may be manipulated by decreasing the number of coils of the ribbon of spring steel 608 on the output drum and/or decreasing the diameter of the storage drum 606, for example.

In some implementations, the storage drum 606 may include a central axis 614. As will be discussed below, the central axis 614 may be attached to the spool thus biasing the rotational force of the spool in a wound position.

In other implementations, a constant-force spring may be used in place of the constant torque spring mechanism described above.

FIG. 7 illustrates a side surface view of reel 100 including medical tubing intake/outtake opening 210. As mentioned above, intake/outtake opening 210 may provide an opening for the medical tubing to exit the reel 100 and, ultimately, interact with a user. In some implementations, intake/outtake opening 210 may be formed by the assembly of an opening on the molding each of the first half 102 and second half 104. In other implementations, the intake/outtake opening 210 may be solely located on the first half 102 or the second half 104. Furthermore, intake/outtake opening 210 may be positioned at any location on reel 100. For example, intake/outtake opening 210 may be positioned on any side of reel 100.

The geometry of intake/outtake opening 210 is illustrated substantially as a rectangle with rounded corners; however, in other implementations, the intake/outtake opening 210 may be formed as any number of suitable shapes (e.g., circular, square, ovoid, octagonal, etc.).

In some implementations, a perimeter of intake/outtake opening 210 may include a thicker molding or lip 702. In some implementation, lip 702 may be thicker than a wall of the first half 102 and/or second half 104. In some implementation, the lip 702 may include an edge guard (not shown) in order to protect and/or reduce friction between the medical tubing extending through the intake/outtake opening 210. In some implementations, the edge guard may include, for example, vinyl, polycarbonate, polyethylene/acrylonitrile butadiene styrene, polychloroprene or the like. In some implementations, each surface of lip 702 may include a roller configured to reduce friction as the medical tubing is retrieved from and retracted to the reel 100.

### Example Spool and Ratchet Mechanism

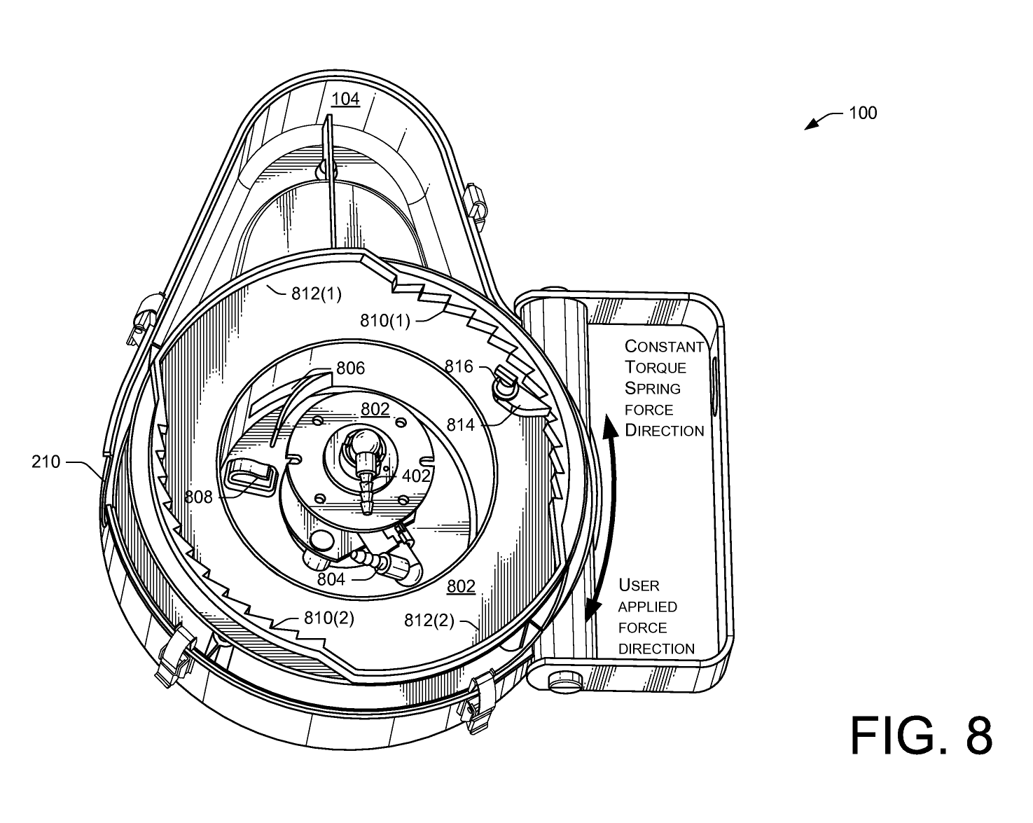

FIG. 8 illustrates a perspective view of reel 100 with first half 102 removed. As illustrated reel 100 may include spool 802. In some implementation, the spool 802 may have a storage portion for storing medical tubing and an internal portion for coupling the medical tubing to the intake connector 402.

In some implementations, the internal portion of spool 802 may include a connector 804. Connector 804 may be configured to couple to a length of medical tubing stored on spool 802. In some implementation, spool 802 may have an internal channel that may supply a substance (e.g., oxygen gas) from the intake connector 402 to the connector 804. In some implementations, the substance may be further pushed through the attached tubing for consumption by a user.

In some implementations, the connector 804 may be interchangeable with other connector configurations to accommodate various diameters and/or types of medical tubing.

Spool 802 may include a tube conduit 806 to allow the medical tubing to pass from the internal portion of spool 802 to the storage portion of spool 802. In some implementations, the tube conduit 806 may be positioned such that the medical tubing passing from the connector 804 may be wound on the storage portion of spool biased toward a direction that spool 802 may rotate while the medical tubing exits reel 100.

Spool 802 may also include a tubing clamp 808 to hold and/or secure the medical tubing as it passes from connector 804 to the tubing conduit 806. In some implementations, tubing clamp 808 may prevent the tubing from disengaging the connector 804 as an entire length of medical tubing is pulled from reel 100. In other implementations, tubing clamp 808 may provide proper alignment for the medical tubing as it enters the tube conduit 806.

In some implementations, the storage portion of spool 802 may hold various lengths and types of tubing such as flexible medical tubing. For example, in some implementation, spool 802 may hold less than 50 feet of flexible medical tubing. In other implementations, spool 802 may hold more than 100 feet of crush-resistant oxygen tubing. A first end the tubing may be attached to connector 804, while in some implementations; a second end of the tubing may be attached to a user or tubing swivel, adapter, leader line, or the like after exiting the intake/outtake opening 210 on reel 100.

FIG. 8 further illustrates that spool 802 may also include multiple clusters of teeth (shown generally as 810(1) and 810(2)) on a top surface of spool 802. FIG. 8. shows two clusters where each cluster includes twelve teeth. It should be noted that this is merely an example implementations and many of other implementations are envisioned. For example, spool 802 may include more clusters of teeth where each cluster includes fewer teeth. In some implementations, each cluster of teeth 810(1) and 810(2) alternate with a toothless space 812(1) and 812(2). In other implementations, where spool include more than two clusters of teeth they are separated by a toothless space.

FIG. 8 also illustrates that the top surface of spool 802 may include pawl 814. In some implementation, pawl 814 and the teeth of clusters 810(1) and 810(2) form a ratcheting mechanism configured to stop a rotation motion of spool 802 relative to reel 100. In some implementations, pawl 814 may include an internal spring within pawl housing 816. Internal spring may be configured to assert a force on pawl 814 such that pawl 814 is biased in direction toward the clusters of teeth 810(1) and/or 810(2). As mentioned above in FIG. 4, in some implementations, pawl housing 816 may be secured by the pawl holder 406 of the first half 102 of reel 100.

In some implementation, pawl 814 may stop backward motion of spool 802 at one or more discrete points (i.e., boundary of each tooth within the clusters 810(1) or 810(2)). In some implementations, the backwards force on the spool 802 may be supplied by the constant torque spring mechanism 602 described above with regard to FIG. 6.

In some implementations, when a user applies a force to the medical tubing stored on the storage portion of spool 802, the spool may move in a direction such that the pawl 814 may interact with the teeth of each cluster. As spool 802 is rotated, medical tubing may be fed from the storage portion of spool 802 through intake/outtake opening 210 on reel 100.

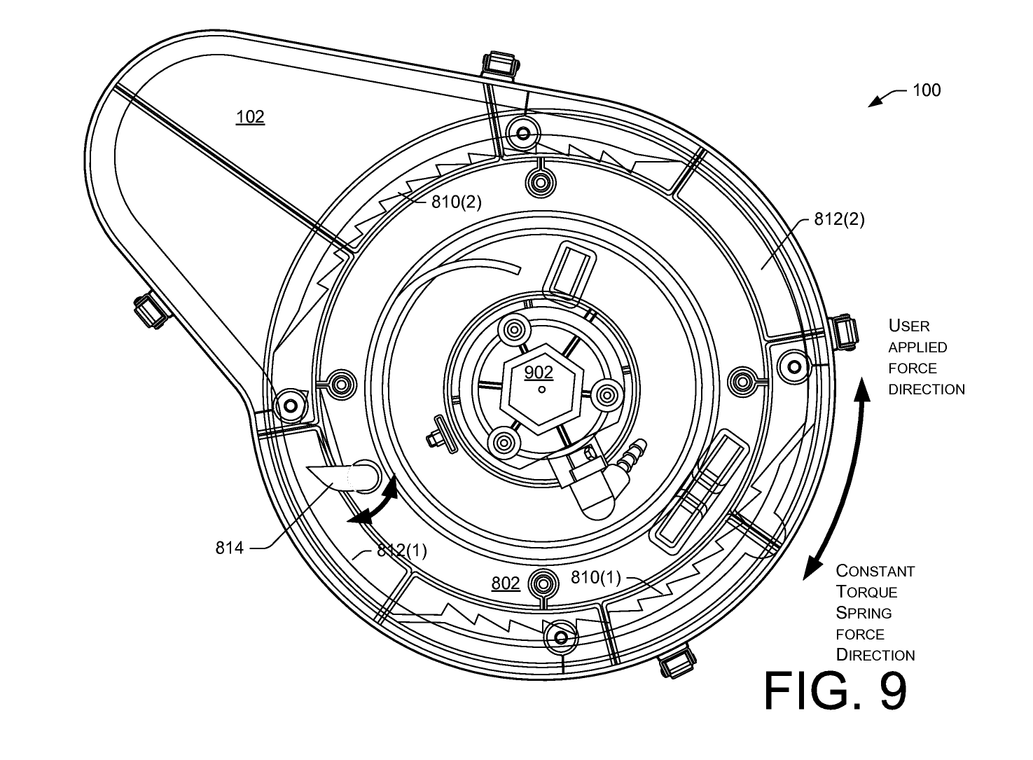

FIG. 9 illustrates a second surface view of reel 100 with second half 104 removed. FIG. 9 further illustrates a transparent spool 802 with ratchet mechanism. Specifically, FIG. 9 illustrates an action of pawl 814 as the pawl 814 passes one of the clusters of teeth.

In some implementations, the internal spring within the pawl housing may allow the pawl 814 to rotate away from the central axis of the spool. In some implementations, when a user applied force on the fed medical tubing ceases and the position of the pawl 814 is within one of the toothless spaces 812(1) or 812(2), the pawl 814 may rotate such that the constant torque spring may spin the spool 802 in a direction to retract medical tubing extending from intake/outtake opening 210. In some implementations, the rounded surface of pawl 814 may pass over each tooth of each cluster 810(1) and 810(2) without catching. However, in other implementations, when a user applied force on the fed medical tubing continues, the pawl 814 may continue on to the teeth of an adjacent cluster.

FIG. 9 also illustrates an inner axis 902 on spool 802. In some implementations, inner axis 902 may be configured to couple to with the central axis 614 of the storage drum 606 of the constant torque spring mechanism 602.

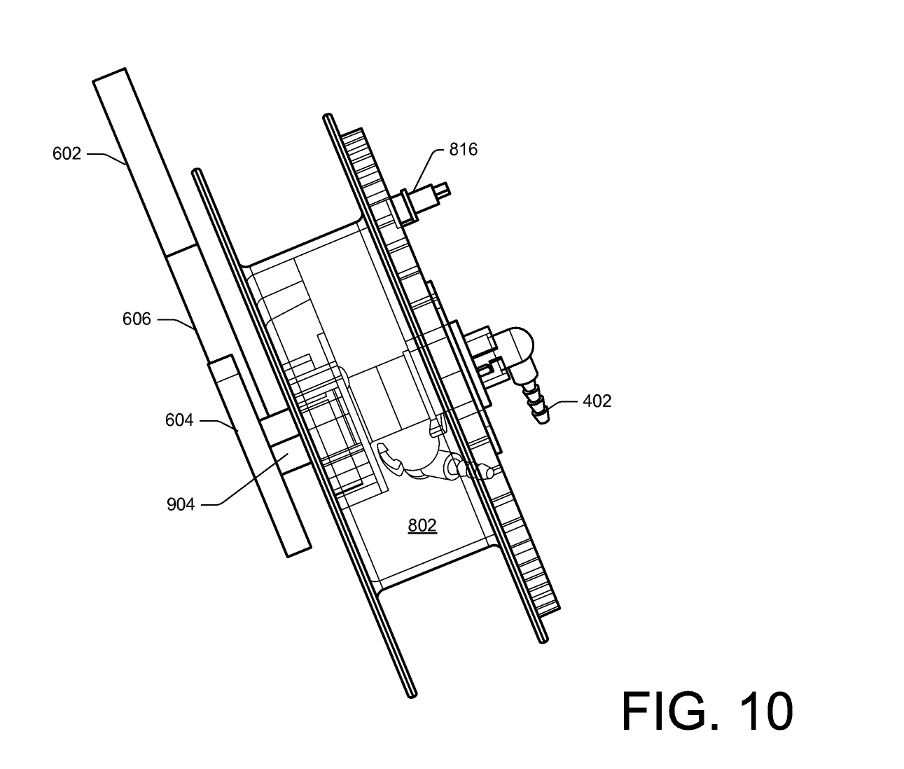

FIG. 10 illustrates a perspective view of the constant torque spring mechanism as coupled to spool 802. As illustrated in FIG. 10, inner axis 902 may provide a link between spool 802 and storage drum 606.

### Alternative Retractable Medical Tubing Reel

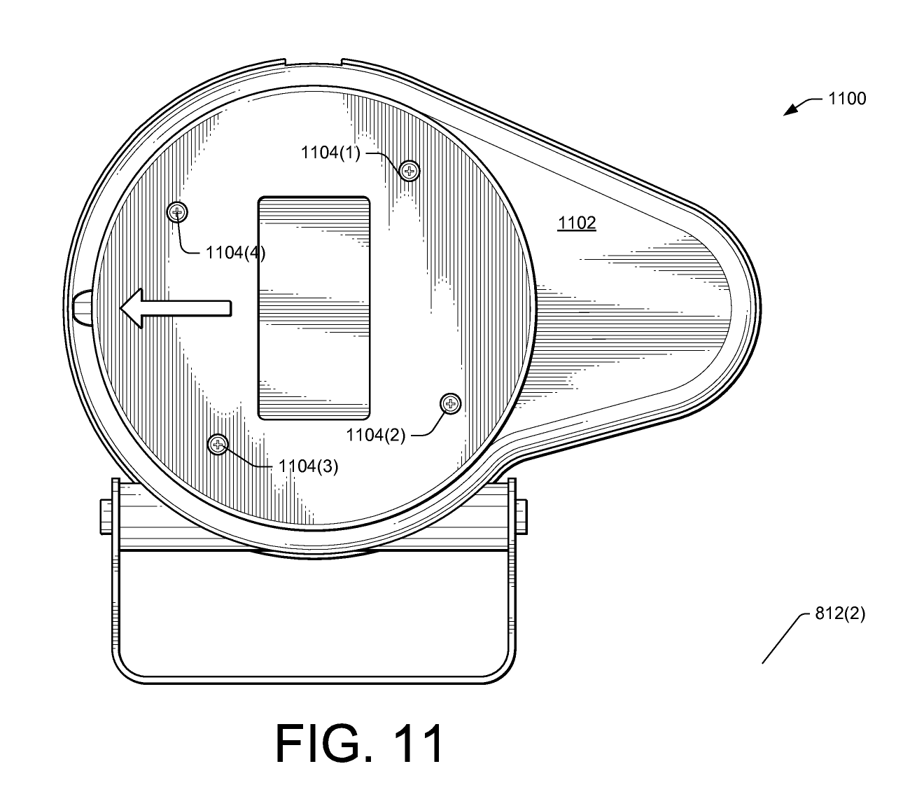

FIG. 11 illustrates a first surface view of first half 1102 of reel 1100 configured without clips (i.e., clips 108(1)-(4) shown in FIG. 2). In some implementations, the first half 1102 of reel 1100 may be form fitted to a second half of reel 1100. In other implementations, the first half 1102 of reel 1100 may be secure to the second half by fasteners 1104(1)-(4). In yet other implementations, other mechanisms may be used to couple the first half 1102 to the second half of reel 1100. (e.g., magnets, snap fit (cantilever type (U-shaped, L-shape, etc.))).

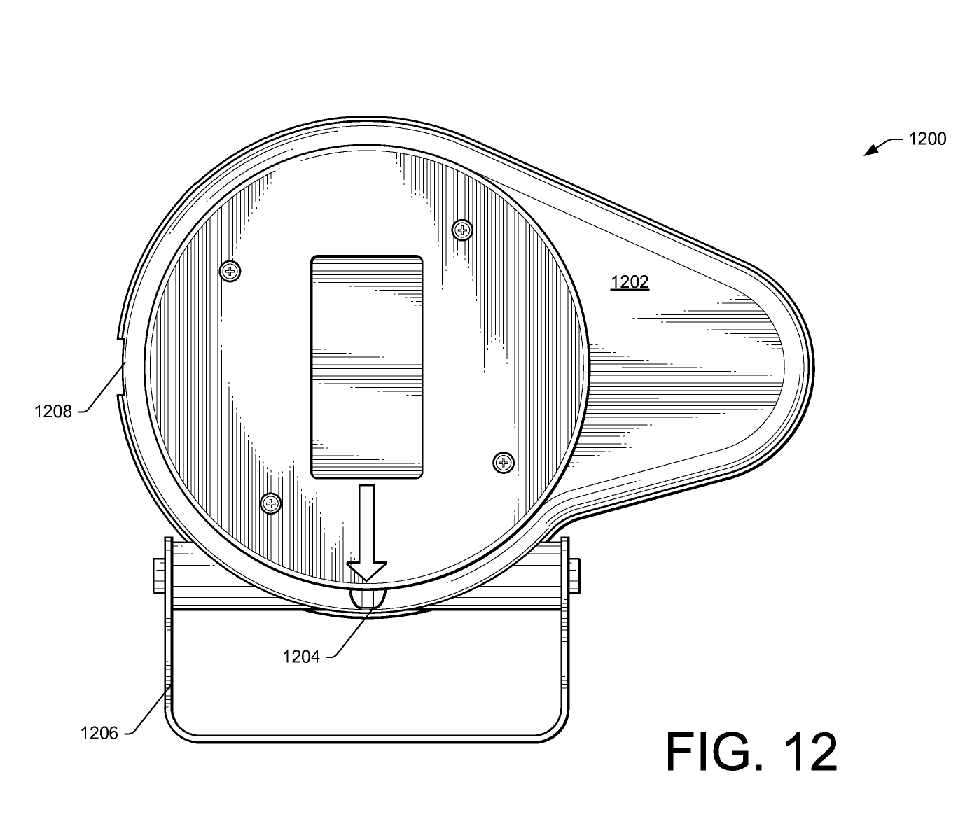

FIG. 12 illustrates a top view of first half 1202 of reel 1200 configured with a tubing intake port 1204 positioned on a side near a mounting bracket 1206. In this implementation, it may be more convenient to attach medical tubing to reel 1200 since the intake port 1204 may be nearer a supply a substance traveling through the medical tubing. For example, the intake port 1204 may be nearer a wall-mounted oxygen source providing oxygen gas to a user through the medical tubing on reel 1200.

FIG. 12 also illustrates a medical tubing outtake opening 1208 positioned substantially on the bottom of reel 1200. In some implementations, intake/outtake opening 1208 may provide an opening for the medical tubing to exit the reel and ultimately couple to a user. Outtake opening 1208 may be implementation in any of the way described above with regard to intake/outtake opening 210 in FIG. 2. Furthermore, in other implementations, intake/outtake opening 1208 may be placed at any position around the perimeter of reel 1200 to provide a suitable exit point for the medical tubing stored in reel 1200.

In yet another implementation, the spool may include a locking mechanism configured to lock a position of the spool relative to the first and second half of the case. In such implementations, the locking mechanism may prevent the spring mechanism from automatically retracting a portion of medical tubing extending from the spool.

### Example Process

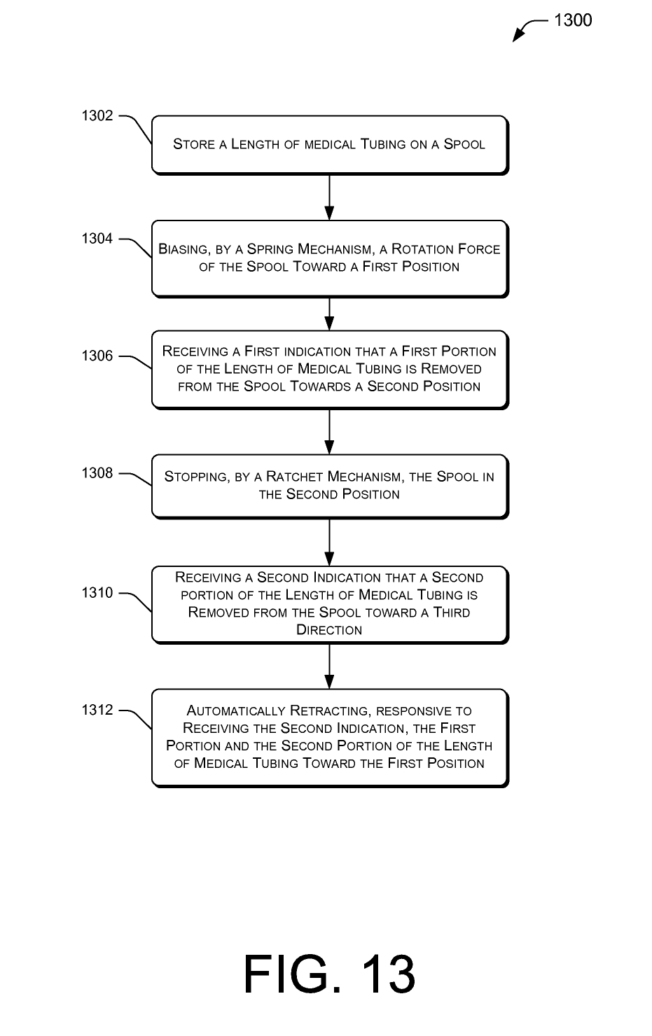

FIG. 13 illustrates an example process 1300 for implementing the techniques described above of automatically retracting a portion of medical tubing using a retractable medical tubing reel. The process 1300 is illustrated as a logical flow graph, each operation of which represents a sequence of operations. The order in which the operations are described is not intended to be construed as a limitation, and any number of the described operations can be combined in any order and/or in parallel to implement the process.

At 1302, a length of medical tubing may be stored on a spool. In some implementations, the length of medical tubing may be greater than or equal 25 feet.

At 1304, a spring mechanism may bias a rotation force of the spool toward a first direction. With reference to FIG. 6, the spring mechanism may be a constant torque spring 602 configured to bias the spool toward a wound position where all of the medical tubing stored on the spool is retracted.

At 1306, a first indication may be received that a first portion of the length of medical tubing has been removed from the spool towards a second position. For example, an indication may be received that a user has pulled a portion of medical tubing out of the intake/outtake opening 210.

At 1308, a ratchet mechanism may stop the spool in the second position. With reference to FIG. 8, pawl 814 may interact with one of the plurality of teeth 810(1) location on the spool.

At 1310, a second indication may be received that a second portion of the length of medical tubing is removed from the spool toward a third direction. In some implementations, such an indication may result from a user making a quick sharp tug on the already fed portion of medical tubing. With reference to FIG. 9, such an indication may position the pawl 814 in a toothless area of the spool, thereby releasing the internal spring within the pawl housing.

At 1312, responsive to receiving the second indication, the first portion and the second portion of the length of medical tubing may automatically retract toward the first position. In some implementations, the spring mechanism may manipulate such retraction. For example, the output drum of a constant torque spring may retake any withdrawn portion of the ribbon of spring steel from the storage drum. This rotation of the storage drum may correspondingly rotate the attached spool to retract any fed portion of medical tubing back within a storage portion of the spool.

### CONCLUSION

Although the subject matter has been described in language specific to structural features, it is to be understood that the subject matter defined in the appended claims is not necessarily limited to the specific features described. Rather, the specific features are disclosed as illustrative forms of implementing the claims

What is claimed is:

1. A reel for supplying medical tubing, the reel comprising:
a case comprising a first half coupled to a second half and configured to house:
a spool for holding a length of medical tubing and comprising:
one or more clusters of a plurality of teeth;
a pawl mechanism configured to interact with the one or more clusters of teeth of the plurality of teeth as medical tubing exits the spool;
a spring mechanism configured to place a rotational force upon the spool to automatically retract the length of medical tubing; and
a bracket configured to couple the case to a surface and configured to allow the case to rotate in at least 180 degrees.

2. The reel as recited in claim 1, wherein the spool further comprise an internal channel configured to transfer a substance from an external substance source to the length of medical tubing held on the spool.

3. The reel as recited in claim 2, wherein the transferred substance comprises at least one of oxygen gas, nitrous oxide, or albuterol sulfate.

4. The reel as recited in claim 1, wherein the spool is configured to hold a length of medical tubing of from about 25 feet to about 100 feet.

5. The reel as recited in claim 1, wherein the first half and the second half of the case are coupled by a plurality of clips, a plurality of screws, a plurality of magnets, a plurality of snap fitting cantilevers or form-fitting.

6. The reel as recited in claim 1, wherein the spring mechanism comprises a constant torque spring with an output drum and a storage drum, the constant torque spring configured to bias the rotational force of the spool toward a wound position.

7. A method of supplying and automatically retracting a length of medical tubing, the method comprising:
storing the length of medical tubing on a spool, the spool comprising:
one or more groups of a plurality of teeth position around a top perimeter of the spool and where a space between each group comprises a toothless area;
constantly biasing, by a spring mechanism, a rotation force of the spool toward a first position;
receiving a first indication that a first portion of the length of medical tubing is removed from the spool toward a second position;
stopping, by a ratchet mechanism, the spool in the second position, the ratchet mechanism comprising:
a pawl configured to secure the spool in the second position by interacting with the one or more groups of the plurality of teeth on the spool while the spring mechanism provides the constant biasing;
receiving a second indication that a second portion of the length of medical tubing is removed from the spool toward a third position, wherein the third position places the pawl of the ratchet mechanism within the toothless area; and
automatically retracting, responsive to receiving the second indication, the first portion and the second portion of the length of medical tubing toward the first position.

8. The method as recited in claim 7, wherein the spool further comprises an internal channel configured to transfer at least one of oxygen gas, nitrous oxide, or albuterol sulfate from an external source to the length of medical tubing stored on the spool.

9. The method as recited in claim 7, wherein the spool is configured to store a length of medical tubing of from about 25 feet to about 100 feet.

10. The method as recited in claim 7, wherein the spring mechanism comprises a constant torque spring with an output drum having coiled ribbons of spring steel configured counter coil onto a storage drum as a portion of the length of medical tubing is removed from the spool.

11. The method as recited in claim 7, wherein the spool, spring mechanism and ratchet mechanism are housed within a case having a first half removably coupled to a second half.

12. The method as recited in claim 11, wherein the first half and the second half of the case are removably coupled by a plurality of clips, a plurality of screws, a plurality of magnets, a plurality of snap fitting cantilevers or form-fitting.

13. The method as recited in claim 7, wherein the pawl comprises an internal spring within a housing configured to bias the pawl in direction toward the one or more groups of the plurality of teeth on the spool.

14. A reel assembly, comprising:
a spool housed within a first half and a second half of a case, the spool configured to store a length of medical tubing and comprising a plurality of projections located on a top external portion of the spool and an apex of each of the plurality of projections substantially faced toward a central axis of the spool;
a pawl mechanism comprising an internal spring configured to bias the pawl toward engagement with the plurality of projections on the spool; and
a constant spring mechanism configured to retract a portion of medical tubing when the pawl mechanism is disengaged from the plurality of projections on the spool.

15. The reel assembly as recited in claim 14, further comprising a bracket configured to couple the case to a surface and allow the case to rotate in at least 180 degrees.

16. The reel assembly as recited in claim 14, wherein the length of medical tubing comprises a continuous length of medical tubing greater than or equal to 25 feet.

17. The reel assembly as recited in claim 14, wherein the spool further comprises an internal portion comprising a tubing clamp configured to secure the medical tubing relative to the spool.

18. The reel assembly as recited in claim 14, wherein the spool further comprises one or more projection-less areas alternating with a plurality of areas having the plurality of projections, and the constant spring mechanism is configured to retract when the pawl mechanism enters one of the one or more projection-less areas.

19. The reel assembly as recited in claim 14, wherein the constant spring mechanism comprises a constant torque spring with an output drum having coiled ribbons of spring steel configured counter coil onto a storage drum as a portion of the length of medical tubing is removed from the spool.

20. The reel assembly as recited in claim 14, further comprising a case having a first half removably coupled to a second half by a plurality of clips, a plurality of screws, a plurality of magnets, a plurality of snap fitting cantilevers or form-fitting.
